(19) United States
(12) Patent Application Publication
(10) Pub. No.: US 2026/0102492 A1
(43) Pub. Date: Apr. 16, 2026

(54) Inhibition of Natural Killer Cell Cytoxocity

(71) Applicant: University of Cincinnati, Cincinnati, OH (US)

(72) Inventors: Trisha Wise-Draper, Ft Mitchell, KY (US); McKenzie Crist, Cincinnati, OH (US)

(73) Assignee: University of Cincinnati, Cincinnati, OH (US)

(21) Appl. No.: 19/116,689

(22) PCT Filed: Sep. 28, 2023
(86) PCT No.: PCT/US2023/034055

§ 371 Date: Mar. 28, 2025

Related U.S. Application Data
(60) Provisional application No. 63/410,819, filed on Sep. 28, 2022.

(51) Int. Cl. A61K 40/15; A61K 31/155; A61K 31/17; A61K 31/341; A61K 39/395; A61K 40/30; A61P 37/04

(52) U.S. Cl. CPC A61K 40/15; A61K 31/155; A61K 31/17; A61K 31/341; A61K 39/3955; A61K 40/30; A61P 37/04

(57) ABSTRACT

A method of inhibiting CXCL1 in one or more natural killer (NK) cells in a subject is provided. The method involves administering one or more pharmaceutically effective doses of metformin to the subject. In another embodiment of the invention, a pharmaceutical composition useful for increasing natural killer cell activity in a subject is provided. The composition includes pembrolizumab, a CXCR2 inhibitor and a pharmaceutically acceptable excipient.

### CROSS-REFERENCE TO RELATED APPLICATIONS

This application is a continuation of PCT Application No. PCT/US23/34055 filed Sep. 28, 2023, which claims the benefit of the filing date of U.S. Provisional Application No. 63/410,819 filed Sep. 28, 2022, the disclosure of which is incorporated by reference herein in its entirety.

### STATEMENT REGARDING FEDERALLY SPONSORED RESEARCH OR DEVELOPMENT

This invention was made with government support under grant TR001426 awarded by the National Institutes of Health. The government has certain rights in the invention.

### FIELD OF THE INVENTION

This invention relates generally to treatments for head and neck cancers.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Head and neck cancers (HNC) are the sixth most common malignancy worldwide. Most cancers of the head and neck are squamous cell carcinoma (HNSCC). Etiologic factors include alcohol and tobacco use, and infection with oncogenic viruses including human papilloma virus (HPV). Over 70% of new HNSCC cancers present as locally advanced disease (LAHNSCC) requiring multi-modality treatment. For patients with locally advanced disease ineligible for resection, concurrent cisplatin-based chemoradiotherapy (CRT) is still recognized as the standard of care treatment. Despite improved outcomes with CRT, there is still a high level of disease recurrence, especially in the HPV-negative, smoking population, necessitating improved treatment regimens.

Treatment combinations involving immunotherapy have emerged as promising options to combat advanced stage disease. Additionally, alternative drug development studies have begun to explore repurposing medications originally intended to treat other non-cancer diseases. Metformin, a biguanide, is FDA approved to treat type II diabetes, polycystic ovary disease, and has recently been examined in cancer clinical trials. In preclinical studies, metformin has been shown to have anticancer effects in solid tumors, mainly through stimulation of the AMP-activated protein kinase (AMPK), and subsequent inhibition of the mechanistic target of rapamycin (mTOR). Furthermore, in HNSCC, it has been shown to prevent the development and progression of oral tumors in mouse models.

Recent evidence suggests metformin may also impact the host immune system by playing a key role in maintaining the multifunctional capacity of tumor infiltrating lymphocytes (TILs), including infiltration and activation of effector memory T-cells (TEM), natural killer cells (NK), and natural killer-like T (NKT) cells in a liver-injury mouse model. Reducing oxygen consumption further potentiated TIL functionality, and, in combination with anti-programmed death 1 (PD-1) signaling blockade, metformin resulted in enhanced tumor growth inhibition. Despite the evidence of the effect of metformin on lymphocytes in the tumor microenvironment (TME), little was known about these effects in peripheral blood mononuclear cells (PBMC) which migrate to the tumor site upon antigen stimulation.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a method of inhibiting CXCL1 in one or more natural killer (NK) cells in a subject is provided. The method involves administering one or more pharmaceutically effective doses of metformin to the subject. In another embodiment of the invention, a method of inhibiting CXCL1 in one or more natural killer (NK) cells in a subject is provided. The method involves administering one or more pharmaceutically effective doses of pembrolizumab and a CXCR2 inhibitor to the subject. In one embodiment, the CXCR2 inhibitor is selected from the group consisting of Navaxarin, AZD5068, and SB225002. In another embodiment, the CXCR2 inhibitor is Navaxarin.

In an embodiment of the invention, a pharmaceutical composition useful for increasing natural killer cell activity in a subject is provided. The composition includes pembrolizumab, a CXCR2 inhibitor and a pharmaceutically acceptable excipient. In one embodiment, the CXCR2 inhibitor is selected from the group consisting of Navaxarin, AZD5068, and SB225002. In another embodiment, the CXCR2 inhibitor is Navaxarin.

In an embodiment of the invention, a genetically modified natural killer cell is provided wherein the cell is deficient in CXCL1. In an embodiment of the invention, a genetically modified natural killer cell is provided wherein the cell is deficient in CXCR2. In an embodiment of the invention, a method of increasing natural killer cell activity in a subject is provided. The method involves administering to the subject a pharmaceutically effective dose of pembrolizumab and one or more genetically modified natural killer cells wherein the genetically modified natural killer cells are deficient in CXCR2.

### BRIEF DESCRIPTION OF THE DRAWINGS

The objects and advantages of the disclosed invention will be further appreciated in light of the following detailed descriptions and drawings in which:

FIG. 1 is a graph showing that HNSCC NK cells secrete more CXCL1 and that metformin inhibits CXCL1 release.

FIG. 2 is a graph showing that exogenous CXCL1 reverses metformin mediated NK degranulation.

FIG. 3A is a western blot showing lysates of HNSCC NK cells treated with metformin, 50 ng CXCL1, or 10 uM CXCR2i (navarixin) for 24 hrs.

FIG. 3B is a pair of graphs quantifying the western blots and showing that metformin increases pSTAT1 and decreases pSTAT3 while CXCL1 increases pSTAT3.

FIG. 4 is a graph showing that head and neck squamous cell carcinoma (HNSCC) natural killer (NK) cells do not require AMPK activation for metformin induced NKCA production.

FIG. 5 is a graph showing that mTOR inhibition enhances perforin production.

FIG. 6 is a graph showing that pSTAT1 is involved in metformin induced NKCA production.

FIG. 7 is a graph showing that pSTAT3 is involved in CXCL1 overcoming metformin induced perforin production.

FIG. 8 is a graph showing that metformin reduces CXCL1 through mTOR and pSTAT3.

FIG. 9 is a graph showing that CXCL1 is decreased in metformin treated patients.

FIG. 10A is a graph showing that CXCL1 is increased with non-responders to Pembrolizumab.

FIG. 10B is a graph showing the plasma data from (A) divided into responder and non-responders.

FIG. 11 is a graph showing that pembrolizumab treatment of head and neck cancer patients results in a trend to higher levels of CXCL1 in the plasma.

FIG. 12A is a graph showing peripheral blood mononuclear cells (PBMCs) that were treated with either pembrolizumab (PD-1 inhibitor), exogenous CXCL1, Navaxarin (CXCR2 inhibitor) or combination and combined with CFSE labeled HNC cells. Cytotoxicity was measured by 7AAD 4 hours after incubation. N=5, PBMCS or NK cells isolated from the same PBMCs from patient samples.

FIG. 12B is a graph showing NK cells that were treated with either pembrolizumab (PD-1 inhibitor), exogenous CXCL1, Navaxarin (CXCR2 inhibitor) or combination and combined with CFSE labeled HNC cells. Cytotoxicity was measured by 7AAD 4 hours after incubation. N=5, PBMCS or NK cells isolated from the same PBMCs from patient samples.

FIG. 13 is a graph showing the percentage of NK cell killed target cells for scrambled, scrambled+CXCL1, NK cells with CXCL1 knocked out and Knock out NK cells with CXCL1.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As used herein, the term “pharmaceutically effective” means an amount sufficient to effect the desired change in the subject.

The term “engineered”, as used herein, refers to a nucleic acid molecule, protein molecule, complex, substance, or entity that has been artificially designed, produced, prepared, synthesized and/or manufactured. Therefore, the engineered product is a non-naturally occurring product.

In one embodiment, the present invention involves the use of metformin to inhibit CXCL1 in natural killer (NK) cells, resulting in enhanced NK cellular cytotoxicity. In one embodiment, the present invention is a method of inhibiting CXCL1 in one or more natural killer (NK) cells in a subject comprising administering one or more pharmaceutically effective doses of metformin to the subject. In addition, it was found that CXCL1 exposure can inhibit NK cell cytotoxicity and that inhibiting CXCR2 (receptor for CXCL1) reverses this effect. In another embodiment of the present invention, a composition that is useful for increasing NK cell cytotoxicity in a subject is disclosed. The composition comprises pembrolizumab and a CXCR2 inhibitor.

### Head and Neck Cancer

In 2023 an estimated 609,000 people died from cancer in the U.S. and 1.1 million were newly diagnosed, costing the United States roughly $150 billion. The immense loss of life, reduction in quality of life for survivors, and financial cost have led to cancer as one of the most studied diseases in the world. Despite all the resources allocated to cancer research and drug development, outcomes have only improved by about 1.5% per year.

Worldwide, approximately 900,000 people are diagnosed with head and neck cancer (HNC) each year, and nearly half will die from the disease. The annual number of cases of HNC are expected to rise 30% by 2030. HNC includes cancers of the oral cavity, salivary gland, pharynx, and larynx and is currently more prevalent in men than women (3:1 diagnosis, respectively). Head and neck squamous cell carcinoma (HNSCC) makes up about 90% of head and neck cancers.

Despite intensive multimodal care, up to 50% of patients will relapse after entering remission and those who survive report lower quality of life, including inability to effectively swallow and speak, increased anxiety, and depression.

### Natural Killer Cells

Natural Killer (NK) cells were first discovered in the 1960s, having formerly been mistaken for a subset of T cells. They are found in blood, bone marrow, tonsils, the spleen, and lymph nodes. Within mice they are known to mature within bone marrow and secondary lymphoid tissues. It is unknown exactly where human NK cells mature. They are a type of lymphocyte, a white blood cell that resides in blood and lymph nodes. Lymphocytes begin as pluripotent stem cells and can become innate lymphoid cells, B-cells, T cells, or NK cells. Activation of the CD132 receptor results in commitment to either T cells or NK cells. Outside signals, such as IL-15 and IL-17, can induce these cells to become NKP cells that express early NK receptors such as NKp46 and NKG2D. Additional receptors and functions arise with continued maturation. There are two major subsets of NK cells: CD56bright, CD16-negative cells, known as cytokine producing NK cells, and CD56dim, CD16+ NK cells, known as cytotoxic NK cells. These subsets can further be divided, such as memory-like NK cells, but this dissertation will focus only on the two main subtypes. Cytokine producing NK cells are vital to whole immune cell function. When responding to tumors and pathogens, these cells produce IFNγ, TNFα, and GM-CSF, which activate T cells. They also secrete chemotactic cytokines called chemokines to recruit other lymphocytes to the infected tissue. Cytotoxic NK cells are part of the body's first line of defense against pathogens and cancer.

Adaptive immune cells recognize self-cells (and therefore do not attack normal cells in the body) by major histocompatibility complex (MHC) I and II. When the surface protein is a mismatch to known cells, the cytotoxic cells engage and exert toxicity. Cancer cells can downregulate MHC to evade T cells, but NK cells have the unique ability to exert cytotoxicity in the absence of MHC. They mainly achieve this through activation of NKG2D. The ligand for NKG2D is often expressed on infected cells, allowing the NK cell to lyse the infected cell and release the pathogen to then be recognized by DCs, macrophages, and T cells for further elimination in the body. NK cells are also able to bypass many signals that cancer cells upregulate to evade the immune system. Attacking these cancer cells can then help the rest of the immune system recognize the offending cells as cancer. Activated NK cells also release recruiting signals that can bring additional immune cells to the site of the tumor. Therefore, ensuring NK cells are functional in the tumor environment can further power the immune system to fight cancer.

### C-X-C Motif Chemokine Ligand 1 (CXCL1)

The present invention involves the discovery that metformin inhibits CXCL1 in HNSCC NK cells and that CXCL1 inhibits NK cell cytotoxicity. Further, CXCR2 blockade inhibits this. C-X-C motif chemokine ligand 1 (CXCL1) is a neutrophil recruiting chemokine expressed mainly by macrophages and mast cells and circulated at low levels during normal conditions. These neutrophils are usually recruited in response to microbial infections. Under-expression of CXCL1 and lack of recruitment causes sepsis, while overexpression and crowding of neutrophils leads to tissue damage. CXCL1 can bind to two receptors: CXCR1 and CXCR2. Both are highly expressed on neutrophils and NK cells. All chemokines can also bind to the Duffy antigen/Chemokine Receptor (DARC), which can nullify the chemokine. Interestingly, CXCR1 is not expressed in mice and normally leads to bursting after activation in neutrophils in humans. CXCR2, on the other hand, promotes proliferation of neutrophils and when mutated constitutively activates CXCL1 secretion. CXCR1 mutations do not change CXCL1 secretion.

CXCL1 has previously been implicated as a poor prognostic factor in many cancers and is known to function as a neutrophil recruiting cytokine. However, inhibition of NK cytotoxicity is a new and novel function of CXCL1. NK cells are an important component of the immune system and decreased NK cells is associated with advanced cancer. NK cellular therapy is now being studied as a potential therapy in several clinical trials.

As part of the present invention, it was found that NK cells are the main contributors to immune-oncologic activity rather than T-cells. Restoration of NK cell function is an emerging interest in cancer immunotherapy. Patients with HNSCC often have impaired tumor immune surveillance, highlighted by increased quantities of regulatory T-cells (Treg) in the TME and impaired functions of T-cells, NK cells, and NKT cells. NK cells play a pivotal role in the anti-tumor innate immune response requiring strong stimulatory signaling by means of activating receptors that recognize stress-induced ligands on the surface of target cells. Immunotherapies designed to increase NK cell functionality have had mixed results, including an antibody to target the killer immunoglobulin-like receptor (KIR). We have found that patients with HNSCC have lower circulating subpopulations of NK cells with reduced functional capacity and expression of NKG2D receptors, which can be partially restored by metformin treatment. Importantly, circulating NK and NKT cells were reduced in our cohort of HNSCC patients. These NK cells exhibited reduced IFN-γ, indicative of suppressed functionality. While insufficient NK cell activity in cancer is thought to be modulated by immunosuppressive mediators such as activation of certain STAT pathways, the correct balance of pathway activation has not yet been elucidated. What is clear, is that a reduction in NK cells is an indicator of poor survival in patients with advanced stage HNSCC, and future therapeutics should target NK cells both directly and indirectly by impacting cytokine and chemokine balance.

### Metformin Treatment

Metformin has been observed to be directly toxic to tumor cells mostly through AMPK activation and mTOR inhibition, but few studies have evaluated the anti-tumorigenic immune response to metformin in solid tumors in patients from clinical trials. As part of the present invention, it was found that although there is no overall difference in white blood cell count (WBC) or absolute lymphocyte count (ALC) after metformin treatment, there was a larger population of TEM cells after metformin treatment. Metformin also partially restored circulating NK and NKT cell populations with increasing expression of NKG2D back to levels seen in healthy controls. Analysis of the cytokine-profiles of patient serum suggested activated anti-tumor activity, highlighted by increasing IL-2 and TNF-α.

Metformin activates AMPK which in turn reduces mTOR, a pathway upregulated and targeted in cancer cells but also important to maturation and function of immune cells. Metformin has also been identified as a direct inhibitor of mTOR and pSTAT3. pSTAT3 upregulation is a negative prognostic factor in many solid tumors, and negatively regulates NK cell functions. A recent publication indicated that mTORC1 inhibition by everolimus decreases NK cell maturation in peripheral NK cells in breast cancer. The impact on tumor infiltrating cells was not explored and they found that despite lower maturation, NK cells had increased cytotoxic activity when mTOR is inhibited. Here, we also show in an ex vivo setting, metformin can rescue cytokine release and cytotoxicity of suppressed PBMCs and tumor infiltrating NK cells and that metformin mediated NK cellular cytotoxicity is dependent on mTOR inhibition but independent of AMPK.

RNA-seq analysis of metformin treated HNSCC patient-derived NK cells revealed potential alterations of pathways outside of AMPK. Interestingly, metformin significantly downregulated CXCL1, which is normally activated by pSTAT3 as well as NFκB and mTOR, in NK cells. CXCL1 is a neutrophil recruiting chemokine that has been implicated as a negative prognosis factor in many cancers and is highly expressed by CD56dim NK cells. CXCL1 may recruit NK cells to a tumor site, but continued exposure results in increased pSTAT3 and NFκB, which can ultimately lead to exhaustion of NK cells. Metformin treatment inhibited pSTAT3 but also activated pSTAT1. Although increased pSTAT3 can lead to exhaustion of NK cells, exhaustion can be reversed by pSTAT1 activation. The addition of exogenous CXCL1 reversed metformin mediated pSTAT3 inhibition and ablated metformin induced cytotoxicity supporting a role for CXCL1 as an important inhibitor of NK cell cytotoxicity through pSTAT3. Without being bound by theory, given metformin induced NK cellular cytotoxicity was mTOR dependent, it is possible that pSTAT1 activation occurs indirectly by mTOR inhibition or possibly directly by activating a STAT1 phosphorylase, as metformin is known to directly inhibit mTOR independent of AMPK. CXCL1 inhibition by metformin could be reversed by both mTOR and pSTAT3 inhibition, indicating those pathways as related to possible NK cell dysfunction. Inhibiting these pathways at the source of CXCL1 activation using a CXCR2 inhibitor recovered metformin induced activation, indicating CXCR2 inhibitors could help activate dysfunctional cells.

Metformin has proven to be a powerful tool for elucidating how NK cells can be activated by balancing several deactivating and activating signals within the cell. NK targeted studies have failed to achieve high and sustained NK activity. We have determined that NK cell activation can be achieved by pSTAT1 activation by metformin, but can be reversed by errant pSTAT3 and mTOR, both of which are activated by NFkB dependent cytokines such as CXCL1. Many immunotherapies increase these NFkB dependent cytokines, and in turn increase both pSTAT3 and pSTAT1. Importantly, blocking the CXCL1 receptor, CXCR2, restores NK cell activity. Combining a CXCR2 inhibitor, which would reduce activity of pathways that inhibit NK cell activity, with immunotherapies that increase positive pathways could be useful for future head and neck cancer treatments.

The data presented below show that metformin increases both the number and activity of peripheral NK cells and rescues HNSCC patient NK cell mediated cytotoxicity at least partially through CXCL1 inhibition revealing a new mechanism by which metformin exerts an anti-tumorigenic effect.

### METHODS

### Human Samples

Peripheral blood and serum were obtained from patients from the phase 2 clinical trial “Phase II investigation of adjuvant combined cisplatin and radiation with pembrolizumab in resected head and neck squamous cell carcinoma” (NCT02641093) at the University of Cincinnati, University of Louisville, University of Michigan, Medical Center of South Carolina, and MD Anderson Cancer Center. Key eligibility criteria for patient inclusion on trial were confirmation by tissue biopsy of locally advanced HNSCC that was resectable. Key exclusion criteria included metastatic disease, or nasopharyngeal carcinoma as the primary tumor site. Blood samples were collected before and after 1-3 weeks of treatment with pembrolizumab prior to surgery. Peripheral blood and serum were obtained from patients from the clinical trial “Combining pembrolizumab and metformin in metastatic head and neck cancer patients” (NCT04414540) at the University of Cincinnati. Key eligibility criteria for patient inclusion on trial were confirmation by tissue biopsy of recurrent and/or metastatic HNSCC. Key exclusion criteria included known history of diabetes requiring insulin, or nasopharyngeal carcinoma as the primary tumor site. Samples were collected before treatment, 1 week after metformin treatment, and 2 weeks after addition of pembrolizumab. Additional ex vivo studies were performed on age matched peripheral blood obtained from IRB approved studies UCCI-UMB-14-01 (IRB #2014-4755) and general specimen collection protocol (IRB #2017-2137) to investigate differences in molecular and immune cell markers compared to clinical outcomes in HNSCC patients and normal healthy controls. The studies were approved by the Institutional Review Board at the University of Cincinnati and were conducted in accordance with Good Clinical Practice guidelines and the Declaration of Helsinki. Written informed consent was received from all participating patients prior to enrollment.

### Cell Lines

Human-derived HNSCC cell lines CAL27 and HN5 were grown and maintained in 1×DMEM high glucose (Corning), 8 mM L-glutamine (Corning), 10% FBS (Omega Scientific), 1% Pen/Strep (Corning) and 1×essential amino acids (Corning). All cell lines were cultured at 37° C. with 5% CO2. Mycoplasma presence was checked every 3 months (MycoProbe, R&D Systems, Minneapolis, MN) and no cases of contamination within these cell lines were reported as of February 2022. Cell lines were STR Profiled confirmed by Cincinnati Children's Hospital Medical Center Cytogenics Labs in May 2020.

### Primary Cell Lines and Tumor Infiltrating Leukocytes (TILS)

Tumor tissue was collected from untreated HNSCC patients in resection surgery. Tissue was cut up and digested in 0.25% trypsin. Half was plated onto irradiated T293 feeder cells to establish primary cell lines in keratinocyte media (DMEM/F12, 24.2 ug/mL Adenine Sigma, 1×non-essential amino acids, 100 ug/mL Primocin, 0.4 ug/mL hydrocortisone Sigma, 5% FBS, 1×Sodium pyruvate, 8.3 ng/mL High Chlorea toxin Sigma, 10 uM rock inhibitor Cayman chemical, 1×ITS Thermo Fisher and 0.05 ug/mL EGF Sigma)) and half was plated into 24 well plates in TIL media (RMPI, 1×Pen/Step, 1×Sodium Pyruvate, 2.5 mL of 7.5% stock solution Sodium Bicarbonate, 0.00025% 2-mercaptoethanal Fisher, 10% human serum Sigma, 6,000 U/mL IL-2 (Peprotech), and 100 ug/mL Primocin) Invivogen. Cells were continuously cultured and split regularly to maintain growth.

### Enzyme-Linked Immunosorbent Assay (ELISA)

For NK cell perforin assays, NK cells were treated with drug for 24 hrs, washed, and co-cultured with Cal27 cells for 4 hours. Supernatant was then collected, snap frozen in liquid nitrogen and stored at −80 until thawed for single use. For patient samples, undiluted plasma was added directly to ELISA. ELISA kit for perforin was obtained from ABCAM. ELISA was performed according to the manufacturer's protocol. Protocols included overnight coating of capture antibody diluted by lot number recommendation, 1 hr block with 400 uL 2% BSA in PBS, 2 hr sample incubation at room temperature of 100 uL sample (no dilution of sample was performed in these experiments), 2 hr capture antibody incubation with 100 uL capture antibody diluted by lot number recommendation, 20 min Streptavidin incubation with 100 uL of 1:40 dilution of stock, and 15 min incubation with 100 uL included TMB ELISA reagent. 50 uL stop solution of in house 2N sodium sulfide was added to stop reaction before reading at 500 nm wavelength on plate reader. All plates included standard curve prepared per lot number recommendation with range of 35-2000 pg.

### Single-Cell Multiplex Cytokine Profiling

PBMCS were thawed and stimulated with 100 U/mL of IL-2 for 16 hrs. PBMCs were prepared per manufacturers protocol for CD56 isolation (Miltenyi Biotec #130-050-400). Isolated CD56+ cells were prepped according to IsoPlexis' Isocade Single Cell Polyfunctional Strength protocol and treated with 12 mM metformin. Approximately 30,000 cells were loaded onto IsoPlexis Single Cell Secretome IsoCode chips (IsoPlexis, Haven, CN) and analyzed with the IsoLight system.

### Natural Killer Cytotoxicity Assay (NKCA)

NK cells were isolated from PBMCs using the EasySep Human NK Cell Isolation Kit (Stem Cell Technologies). Cells were washed in Robobuffer and treated with drug indicated in legend for 24 hrs in culture medium. One day before co-culture, cells were collected, washed, and stained with 5(6)-Carboxyfluorescein diacetate N-succinimidyl ester (CFDA-SE) (StemCell Technologies). Untreated UMSCC47, Cal-27, or matched primary HNC tumor cell lines cells were plated at 20,000 cell/ml and assumed to double overnight. NK cells were resuspended in RPMI 1640 (Corning) with 5% human serum at a density of 200,000 cells/ml. Cells were co-cultured for a Target:Effector ratio of 1:5 for 4 hrs, collected, and washed in flow buffer. Cells were then stained with 7AAD Viability Staining Solution (Biolegend). Cells were immediately run on a 4-laser BD Instrument (University of Cincinnati Cancer Cell Biology Department, Cincinnati, Ohio). Target cells were considered any cell that was CDFA+. Any 7AAD+ cells were considered dead cells. NK killed cells were calculated by the following equation: (% CSFE+7AAD+experimental co-culture)−(% CSFE+7AAD+control no co-culture).

### RNA-Seq

HNC NK cells were isolated as above and were treated with vehicle or 12 mM metformin for 24 hrs. Cells were washed, suspended in CyroStor, and frozen. Samples were sent to Genewiz/Azenta for standard RNA-seq profiling. Sequence reads were aligned to the current reference mouse genome (GRCh38) using the STAR aligner (43,44) and the reads aligned to each known gene were counted based on the latest GENCODE definitions of gene features.

### Western Blot Analyses

NK92, Cal27, UMSCC47 and HN5 cells were cultured with drug as described. Cells were collected in RIPA (0.05% sodium deoxycholate, 150 nM NaCl, 50 nM Tris HCL, 0.1% SDS, 1% NP-40) and protein content was analyzed by Pierce BCA kit (Thermo 23225). 50 ug of protein was diluted in 1×loading buffer (SDS, bromophenol blue, 47% glycerol, Tris 0.5M pH 6.8, 0.2 mM DTT-G) and heated to 90° C. for 3 minutes. Prepared lysate was loaded into gradient gels (Biorad 4561093) and run at 85V for 1.5 hour in Biorad casing with a Biorad powerpack in 1×running buffer (Tris, Glycine, SDS). Gels were transferred on nitrocellulose and run at 100V on bench for 1 hour in 1×transfer buffer (Tris, Glycine). Membranes were blocked in 5% BSA in TBS for 1 hr, washed, and incubated in primary diluted in 2% BSA in TBS overnight. Membranes were washed and incubated in Licor secondary (Licor 926-32211 and 925-68070) in 5% BSA in TBS for 1 hr. Membranes were washed and imaged on a Licor Odyssey Clx. Images were analyzed in Image Studio Lite V 5.2.

### Statistics

An Unpaired 2-tailed T Test With Welch's correction was used for analysis between flow cytometry in controls versus HNSCC patients. A 2-tailed Student's t test was used to compare differences between HNSCC patients before and after treatment in flow cytometry and cytokine experiments. Differences in immune cell populations between three groups were compared by one-way ANOVA with specific post-hoc contrasts. NKCA data were compared using a paired t test. Statistical analysis was performed in GraphPad Prism (V10). Differences between groups were considered statistically significant when P<0.05.

### PBMC Isolation and Storage

Peripheral blood from consented HNSCC patients or healthy controls was obtained from the University of Cincinnati College of Medicine. Peripheral blood was received in 10 mL EDTA tubes and 5 mL SST tubes. EDTA samples were processed to isolate PBMCs using Ficoll-Paque PLUS (GE-Healthcare Life Sciences) density gradient centrifugation in SepMate tubes (StemCell Technologies). PBMCs were cryopreserved in Cryostor CS10 (Stem Cell Technologies) and stored in liquid nitrogen. SST samples were spun at 1200×g for 15 min. Serum was collected into microcentrifuge tubes and stored at −80 C.

### Drug Treatments

Metformin (13118) and BP-1-102 (28368) were obtained from Caymen Chemicals. Dosomorphin (S7306), Everolimus (S1120), MHY1485 (S7811), and Flubarabine (S1491) were obtained from Selleck Chemicals. Navarixin (HY-10198) was obtained from Medchem Express.

### EXAMPLES

### Example 1

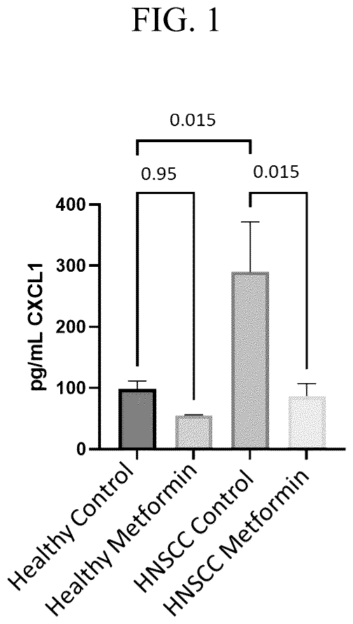

To determine if head and neck squamous cell carcinoma (HNSCC) natural killer (NK) cells produced higher levels of CXCL1 compared to healthy patients and to observe the effect of metformin treatment on NK cells ex vivo, an analysis of NK cells was conducted. Healthy donor and HNSCC NK cells were isolated from PBMCs and were treated for 24 hrs with vehicle or metformin. Supernatant was collected and analyzed by ELISA (analyzed by one way ANOVA. n=10). The results are shown in FIG. 1. It was observed that HNSCC NK cells secreted significantly more CXCL1 than normal NK cells and metformin significantly reduced CXCL1 supernatant levels in HNSCC patient-derived NK cell culture.

### Example 2

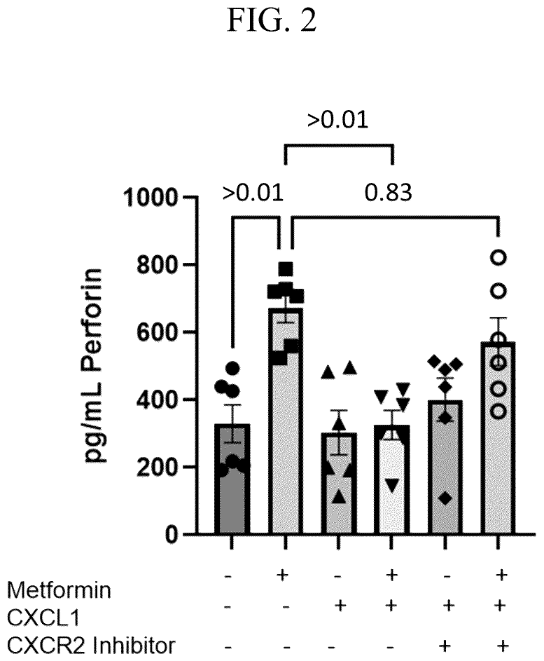

HNSCC patient-derived NK cells were treated with exogenous CXCL1 and an inhibitor of the CXCL1 receptor, CXCR2, or metformin. Patient-derived NK cells were exposed to Cal27 cells for 4 hours and supernatant was collected for perforin analysis by ELISA. Specifically, HNSCC NK cells derived from PBMCs were treated with 50 ng CXCL1, 12 mM metformin, or 10 nM CXCR2i (navarixin) and subjected to perforin against Cal27 target cells. Cells were analyzed by flow and analyzed by one way ANOVA. n=8. The results are shown in FIG. 2.

Although metformin increased perforin as expected, CXCL1 reduced NK cell perforin production. The CXCR2 inhibitor alone and in combination with metformin reversed CXCL1 mediated inhibition.

### Example 3

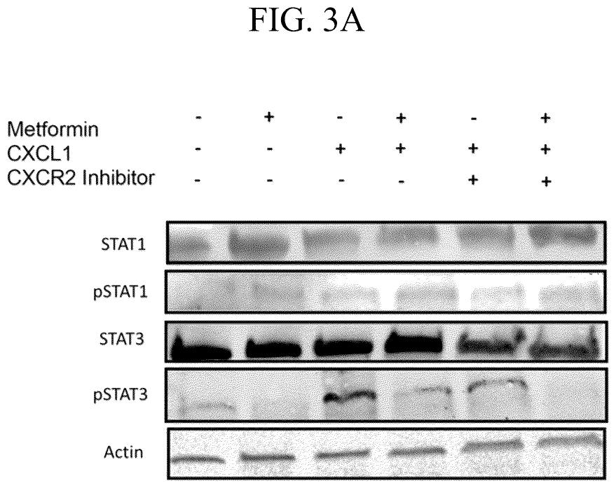

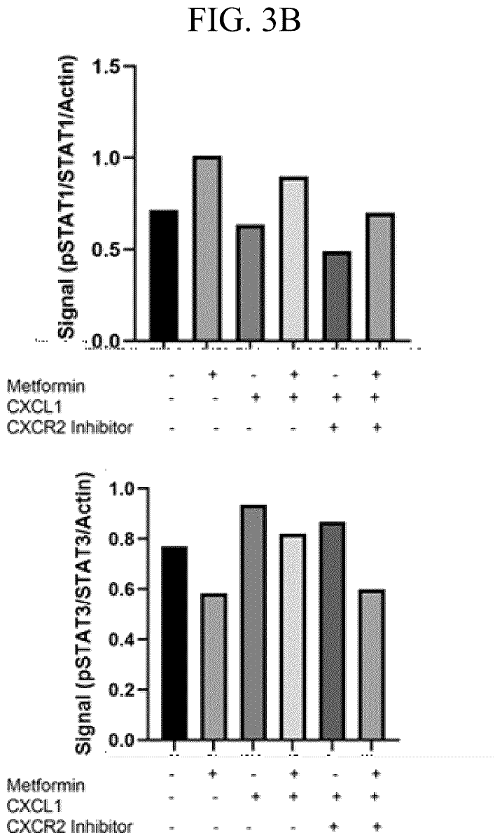

Metformin and CXCL1 could be affecting NK cells in opposing pathways. To determine what those pathways could be, pathways known to be affected by both metformin and CXCL1 were investigated. pSTAT3 and mTOR are known to be increased by activation of the CXCR2 receptor, and mTOR activation can inhibit pSTAT1 expression. Metformin is also hypothesized to inhibit pSTAT3 and mTOR in NK cells. Therefore, HNSCC patient-derived NK cells treated with metformin, CXCL1, or CXCR2i (navaxarin) were collected and analyzed by western blot analysis. Head and neck squamous cell carcinoma (HNSCC) natural killer (NK) cells were treated with metformin, 50 ng CXCL1, or 10 uM CXCR2i (navarixin) for 24 hrs and collected for lysates analyzed by western blot analysis (FIG. 3A). Quantification of western bands is shown in FIG. 3B.

An increase in pSTAT1 was observed with metformin treatment, but CXCL1 did not reverse pSTAT1 expression with metformin or change it from baseline. However, metformin reduction of pSTAT3 was reversed by exogenous CXCL1.

### Example 4

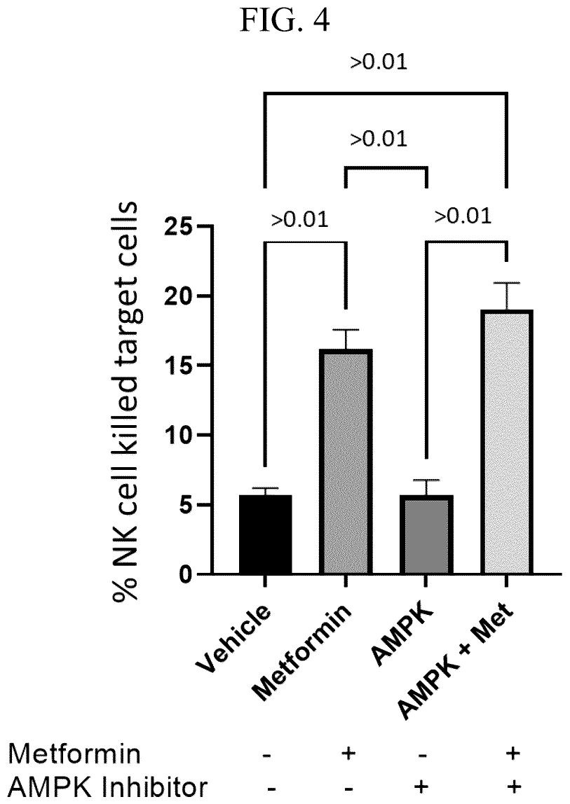

AMPK is known to be upregulated in response to metformin, but studies have indicated that NK cells may be activated independent of AMPK with metformin treatment. To determine if this trend is followed in HNSCC NK cells, we treated HNSCC patient-derived NK cells with vehicle, metformin, or AMPK inhibitor dorsomorphin and analyzed perforin production after exposure to Cal27 cells. Specifically, HNSCC NK cells were treated with vehicle, metformin, or 10 uM AMPK inhibitor (Dosomorphin) for 24 hrs, washed, and co-cultured with Cal27 cells for 4 hrs. Supernatant was collected for ELISA and analyzed by one way ANOVA. n=5. (FIG. 4). Inhibiting AMPK did not change basal perforin or perforin increased by metformin.

### Example 5

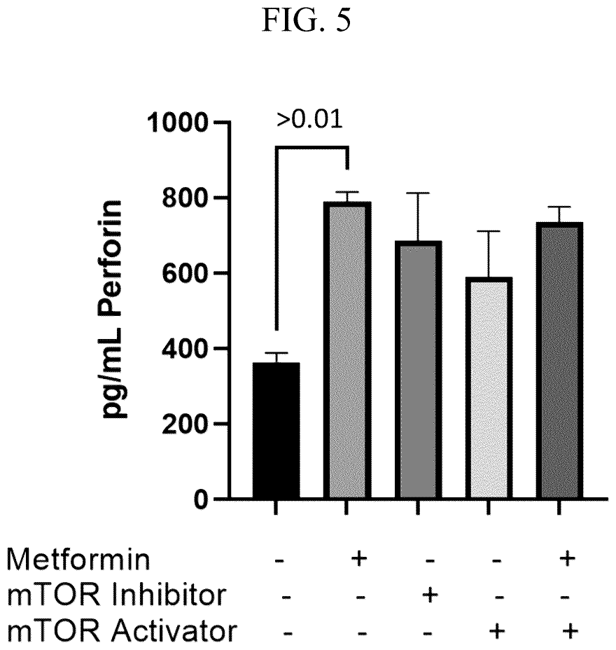

AMPK activation in turn inhibits mTOR although metformin can also inhibit mTOR directly. Given AMPK did not impact metformin induced perforin, it was determined if mTOR inhibition was necessary. HNSCC patient-derived NK cells were treated with vehicle, metformin, 10 uM mTOR inhibitor (Everolimus), or 10 uM mTOR activator (MHY1485) for 24 hrs, washed, and co-cultured with Cal27 cells for 4 hrs. Supernatant was collected for ELISA and analyzed by one way ANOVA. n=5. Inhibiting mTOR enhanced perforin production similar to metformin (FIG. 5) but the combination did not increase perforin over either agent alone. Interestingly, mTOR activation did not result in suppression of perforin.

### Example 6

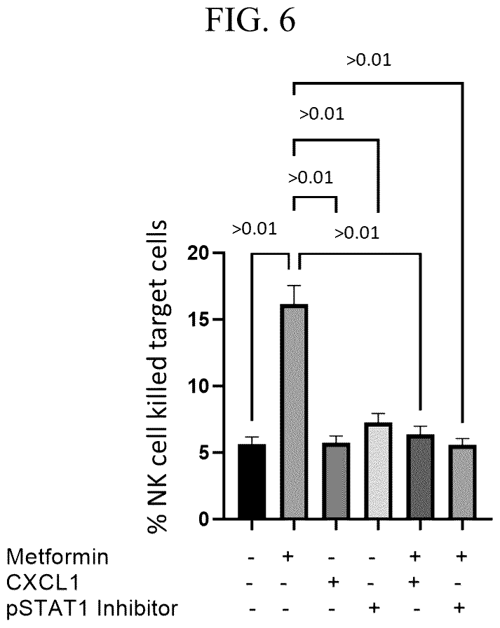

The downstream pathways affected by metformin observed in our western blot analysis (FIG. 3A) were studied. As pSTAT1 can modulate perforin production, HNSCC patient-derived NK cells were treated with vehicle, 12 nM Metformin, or 10 uM of pSTAT1i inhibitor Fludarabine for 24 hrs, washed, and co-cultured with Cal27 cells for 4 hrs. When co-cultured with Cal27 cells, pSTAT1-inhibited NK cells had lower perforin that could be rescued to baseline by metformin (FIG. 6).

### Example 7

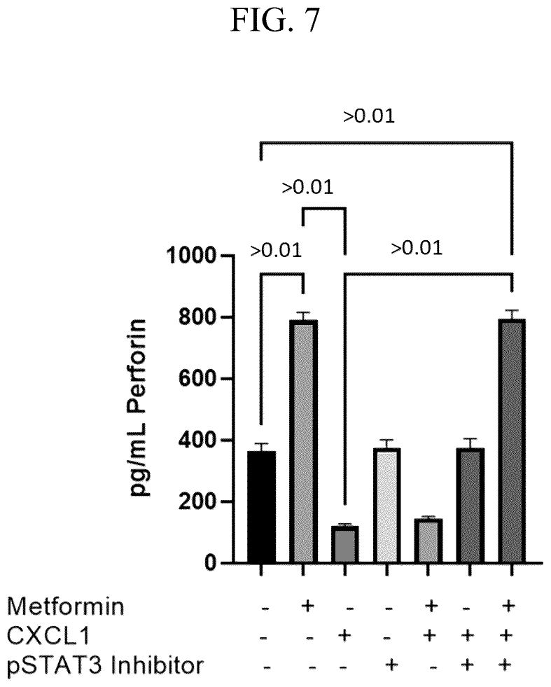

We next determined a potential role for pSTAT3 inhibition for metformin-mediated activation and CXCL1 suppression of perforin. HNSCC NK cells were treated with vehicle, 12 mM metformin, 50 ng CXCL1, or 10 uM pSTAT3 inhibitor BP-1012 and co-cultured with Cal27 cells for 4 hrs. Supernatant was collected for ELISA and analyzed by one way ANOVA. n=5. NK cells treated with a pSTAT3 inhibitor had no effect on perforin at baseline, but STAT3 inhibition was able to rescue CXCL1 mediated suppression of perforin both alone or in presence of metformin (FIG. 7).

### Example 8

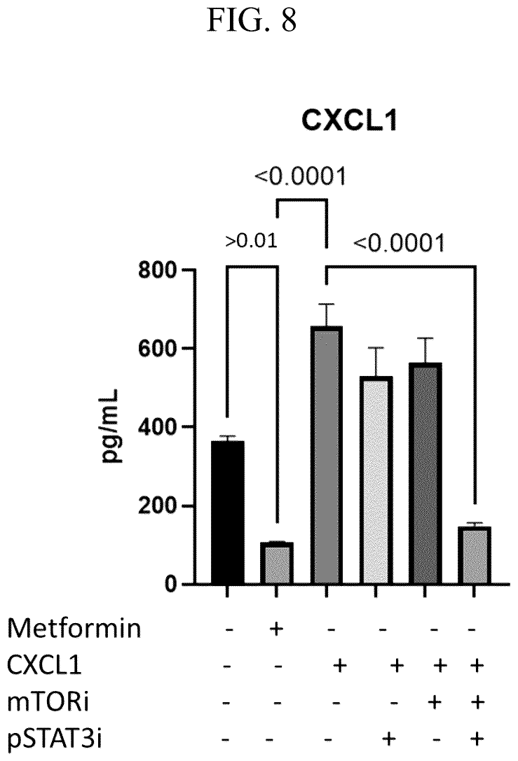

A test was conducted to analyze how metformin was reducing CXCL1 secretion. It was hypothesized that inhibiting mTOR and pSTAT3 in combination would result in a similar reduction of CXCL1 as metformin. HNSCC NK cells were isolated from PBMCs and treated with vehicle, 12 mM metformin, 50 ng CXCL1, 10 uM everolimus, or 10 uM BP-1-102 for 24 hr. The cells were washed and plated in fresh media. After 24 hrs, supernatant was collected for ELISA. Analyzed by one way ANOVA. n=5. pSTAT3 or mTOR inhibition alone did not reduce CXCL1, but a combination decreased levels down to those similar of metformin even in the presence of CXCL1 (FIG. 8).

### Example 9

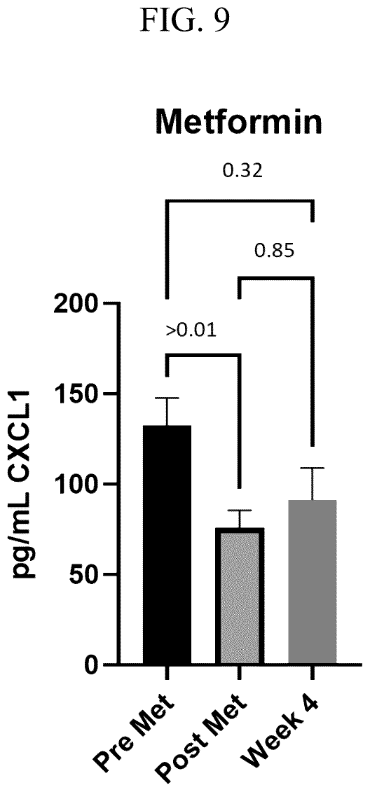

Plasma was taken from patients enrolled in the clinical trial “Combining pembrolizumab and metformin in metastatic head and neck cancer patients” (NCT04414540) and probed for CXCL1 by ELISA. It was analyzed by paired One-Way ANOVA. n=5 (FIG. 9). Samples were collected from patients at 3 time points: (1) pre-treatment, (2) post-metformin only, and (3) post-metformin and pembrolizumab combination (week 4). Metformin treatment reduced circulating CXCL1 as expected, but this decrease was not retained through pembrolizumab treatment.

### Example 10

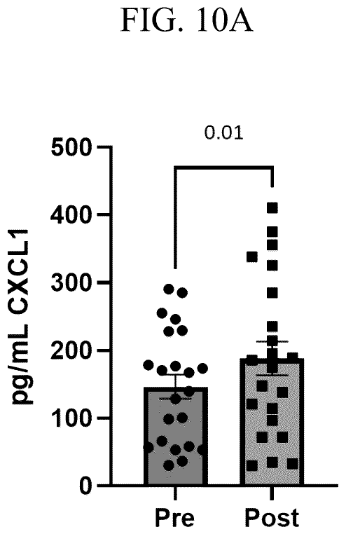

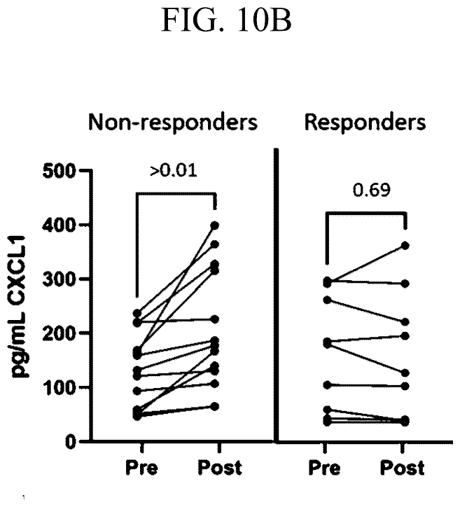

Plasma was taken from the clinical trial “Adjuvant Cisplatin and radiation with pembrolizumab in resected head and neck squamous cell carcinoma” (NCT02641093) and probed for CXCL1. FIG. 10A shows patient plasma pre and post pembrolizumab as subjected to CXCL1 ELISA. FIG. 10B shows plasma data from (A) divided into responder and non-responder. (A) and (B) were analyzed by paired student t test. n=10 responders and 10 non-responders

There was a significant increase in CXCL1 post-pembrolizumab, but when patients were divided out into responders and non-responders, non-responders accounted for the increase. Pre-treatment CXCL1 had no prognostic value (data not shown). This change with pembrolizumab treatment indicates that pembrolizumab increased CXCL1 in patients who do not illicit a response to pembrolizumab.

### Example 11

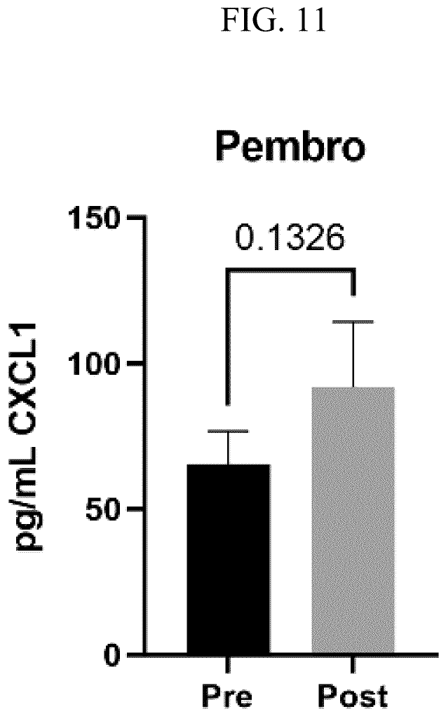

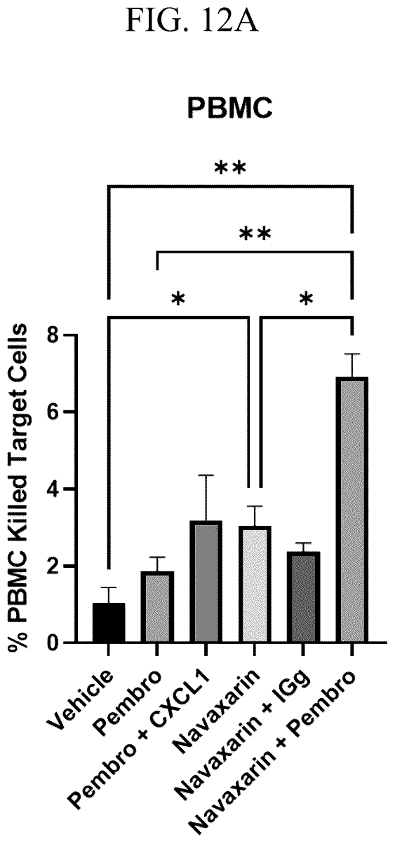

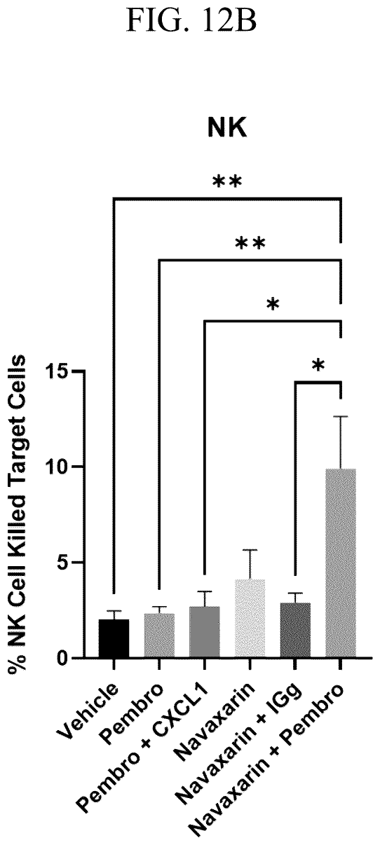

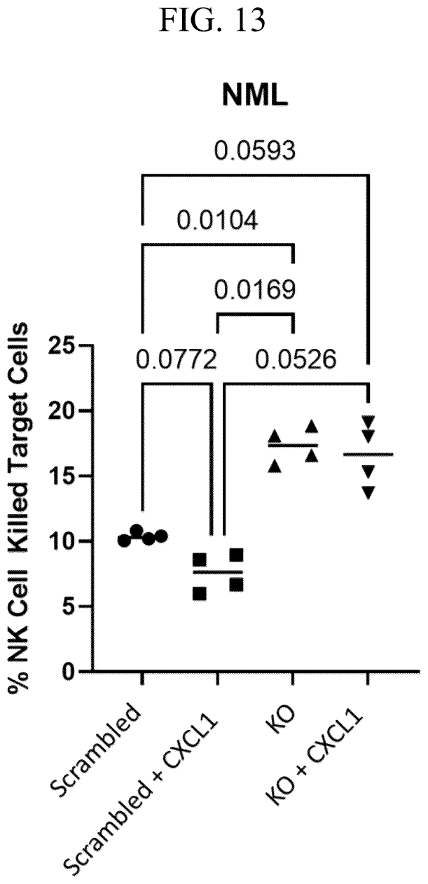

CXCL1 was knocked out by CRISP-Cas9 in NK cells as a novel approach in order to enhance their tumor cytotoxicity. These NK cells can be used as an “off the shelf” anti-cancer therapeutic. NK cells do not require matching of HLA like T cells used for other therapeutics such as CART making our approach more conducive to an increased number of patients. In addition, CXCL1 functions were inhibited with a CXCR2 inhibitor as an alternative treatment strategy with and without addition of a PD-1 inhibitor. Preliminary data suggests synergy with this combination (FIG. 11) as patients treated with pembrolizumab (PD-1 inhibitor) appear to have higher levels of CXCL1 in their plasma from baseline. In addition, the combination of pembrolizumab and CXCR2 inhibitor (Navaxarin) increases both PBMC and NK cell cytotoxicity (FIGS. 12A, 12B and 13).

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method of inhibiting CXCL1 in one or more natural killer (NK) cells in a subject comprising administering one or more pharmaceutically effective doses of metformin to the subject.

2. A method of inhibiting CXCL1 in one or more natural killer (NK) cells in a subject comprising administering one or more pharmaceutically effective doses of pembrolizumab and a CXCR2 inhibitor to the subject.

3. The method of claim 2 wherein the CXCR2 inhibitor is selected from the group consisting of Navaxarin, AZD5068, and SB225002.

4. The method of claim 2 wherein the CXCR2 inhibitor is Navaxarin.

5. A pharmaceutical composition useful for increasing natural killer cell activity in a subject, the composition comprising pembrolizumab, a CXCR2 inhibitor and a pharmaceutically acceptable excipient.

6. The composition of claim 5 wherein the CXCR2 inhibitor is selected from the group consisting of Navaxarin, AZD5068, and SB225002.

7. The composition of claim 5 wherein the CXCR2 inhibitor is Navaxarin.

8. A genetically modified natural killer cell wherein the cell is deficient in CXCL1.

9. A genetically modified natural killer cell wherein the cell is deficient in CXCR2.

10. A method of increasing natural killer cell activity in a subject comprising administering to the subject a pharmaceutically effective dose of pembrolizumab and one or more genetically modified natural killer cells wherein the genetically modified natural killer cells are deficient in CXCR2.
